(19) United States
(12) Plant Patent Application Publication
(10) Pub. No.: US 2013/0326780 P1
(43) Pub. Date: Dec. 5, 2013

(54) Camellia plant named 'Pink Cascade'

(50) ;

(71) Applicant: Shanghai Botanical Garden, The People's Republic China, Shanghai (CN)

(72) Inventors: Jian-Guo Fei, Shanghai (CN); Shu-Cheng Feng, Shanghai (CN); Ya-Li Zhang, Shanghai (CN); Jian-Bin Mo, Shanghai (CN)

(21) Appl. No.: 13/986,736

(22) Filed: May 30, 2013

(30) Foreign Application Priority Data
May 31, 2012 (CN) 20120074

(51) Int. Cl. A01H 5/00

(52) U.S. Cl. PLT/244

(57) ABSTRACT

A new and distinct Camellia L. plant named ‘Pink Cascade’, characterized by its lanceolate leave shape, the width of its leaves measuring at about 1.8 cm to about 2.2 cm, its cascade stem shape, its flower stage at early March to early April, its light pink flower color, its single flower shape, and its diameter of the flower measuring at about 2 cm to about 4 cm.

### LATIN NAME OF THE GENUS AND SPECIES OF THE PLANT CLAIMED

Camellia L.

### VARIETY DENOMINATION

‘Pink Cascade’

### RELATED APPLICATIONS

This application claims the benefit of People's Republic of China application for protection of new varieties plants in China Application Serial No. 20120074 filed on May 31, 2012 under 35 USC Sec. 119(f), hereby specifically incorporated by reference in its entirely.

### BACKGROUND OF THE INVENTION

The present invention relates to a new and distinct cultivar of Camellia plant, botanically known as Camellia L. ‘Pink Cascade’ and will be referred to hereinafter by its cultivar name, ‘Pink Cascade’.

‘Pink Cascade’ wad derived from a breeding program by the inventors at Shanghai Botanical Garden by applying pollen from a Camellia parvi-ovata tree to emasculated flowers of a Camellia japonica ‘Kuro-tsubaki’ tree in the winter of 1996.

During 1997 to 2001, the seeds derived from the cross were planted and screened until the first flower to obtain favorable seedlings. From 2001 to 2011, the favorable seedlings were propagated by cuttings for five times and the stability tests were carried on before a variety was finally selected and named Pink Cascade in 2011.

After hybridization of the parents, the variety was planted and asexual reproduction of the new cultivar was first accomplished by asexual propagation for five generations and there was no genetic variation, therefore its traits are stable.

### SUMMARY OF THE INVENTION

The following traits have been repeatedly observed and are determined to be the characteristics of the new cultivar of Camellia. These attributes in combination distinguish ‘Pink Cascade’ as unique from and all other varieties of Camellia known to the Inventors.

1. The shape of leaves of the ‘Pink Cascade’ is lanceolate.

2. The width of the leaves of ‘Pink Cascade’ measures at about 1.8 cm to about 2.2 cm, and the length of leaves measures at about 5.0 cm to about 8.2 cm.

3. The stem shape of the ‘Pink Cascade’ is cascade.

4. The flower stage of the “Pink Cascade” is about early March to about early April.

5. The flower color of the ‘Pink Cascade’ is light pink in color.

6. The flower shape of the ‘Pink Cascade’ is single.

7. The diameter of the flowers of the ‘Pink Cascade’ is about 2 cm to about 4 cm.

‘Pink Cascade’ can be compared to one parent Camellia japonica ‘Kuro-tsubaki’. Plants of Camellia japonica ‘Kuro-tsubakr ’ differs from the new camellia ‘Pink Cascade’ in that the Camellia japonica ‘Kuro-tsubakr’ has straight stem shape, blackish-red flower color, semi-double flower shape, oval leaf shape, and the diameter of Camellia japonica ‘Kuro-tsubakr ’ flower is about 6 cm to about 8 cm.

Of the many cultivars known to the present inventors, the most similar in comparison to the new Camellia ‘Pink Cascade’ is the Camellia ‘Xiao Fenyu’ (registered for protection of new varieties of plants in China, registration no. 20120001). Plants of Camellia ‘Xiao Fenyu’ differs from the new Camellia ‘Pink Cascade’ in that Camellia ‘Xiao Fenyu’ has oval leaf shape, straight stem shape, the length of leaf is about 6.5 cm to about 7.5 cm, the width of leaf is about 3 cm to 3.5 cm, and the flower stage is about late February to about April.

The comparison between the new cultivar of Camellia ‘Pink Cascade’ and the parent(s) and the other closest comparable plant Camellia ‘Xiao Fenyu’ can be further illustrated by Table 1 below.

TABLE 1

| Denomination | Characteristic in | | | | |
|---|---|---|---|---|---|
| of similar | which the similar | The expression of the characteristic | | | |
| variety | variety is different | Similar variety | | Candidate variety | |
| Xiao Fenyu | Shape of leaf | Oval | | Lanceolate | |
| | Length of leaf | 6.5-7.5 | cm | 5.0-8.2 | cm |
| | Width of leaf | 3-3.5 | cm | 1.8-2.2 | cm |
| | Stem shape | Straight | | Cascade | |
| | Flower stage | Late February to | | Early March to | |
| | | April | | early April | |
| Camellia japonica | Stem shape | Straight | | Cascade | |
| ‘Kuro- | Flower color | blackish-red | | Light pink | |
| tsubaki’ | Flower shape | Semi-double | | Single | |
| | Diameter of flower | 6-8 | cm | 2-4 | cm |

| | | |
|---|---|---|
| Shape of leaf | Oval | Lanceolate |

### BRIEF DESCRIPTION OF THE DRAWINGS

The accompanying colored photographs illustrate the overall appearance and distinct characteristics of the new camellia ‘Pink Cascade’ showing the colors as true as is reasonably possible with colored reproduction of this type. These photos were taken in 2011 and 2013 in China. Colors in the photographs may differ slightly from the color values cited in the detailed botanical description which accurately describes the color of ‘Pink Cascade’.

FIG. 1 provides a side view prospective of a typical tree type of seedling which was finally named ‘Pink Cascade’.

FIG. 2 provides a side view prospective of a typical tree type of ‘Pink Cascade’ which was propagated by cuttings.

FIG. 3 provides close-up view of the leaves of the ‘Pink Cascade’.

FIG. 4 provides close-up view of the flowers of ‘Pink Cascade’.

FIG. 5 provides a side view prospective of plant ‘Pink Cascade’ in flowering stage.

### DETAILED BOTANICAL DESCRIPTION

The following is a detailed description of the new cultivar ‘Pink Cascade’. The phenotype of the new cultivar may vary with variations in environment such as temperature, light intensity, day length, fertilizers and humidity without any change in the genotype of the plant. The descriptors below were taken from 2011 to 2013.

The aforementioned photograph, together with the following observations, measurements and values describe the new Camellia ‘Pink Cascade’ as grown in Shanghai, China. It is planted in the same manner as other common outdoors camellia cultivar, and it grows in area where absolute minimum temperature is above −5 ° C. ‘Pink Cascade’ likes warm and humid climate, prefers acidic soil, and grows well in neutral and light alkaline soil.

The range of day-time growing temperatures is the same with other common outdoors Camellia cultivar. ‘Pink Cascade’ grows well in high temperature environment at summer in Shanghai.

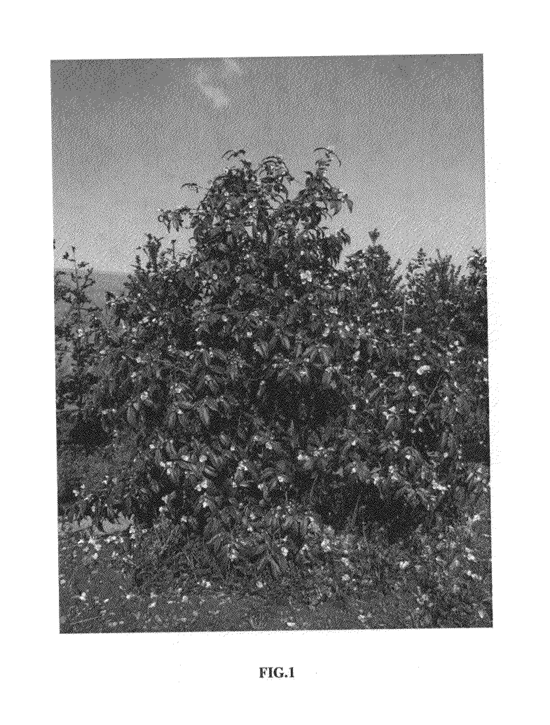

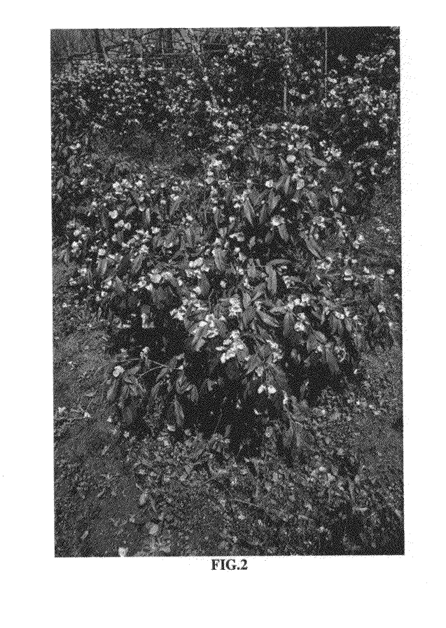

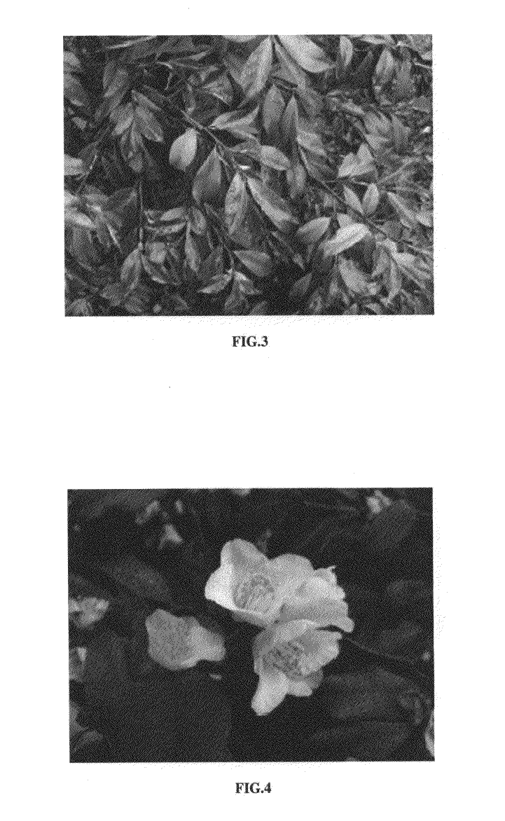

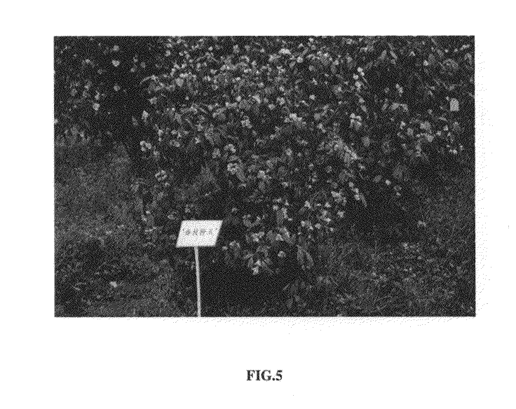

The range of night-time growing temperatures is the same with other common outdoors Camellia cultivar.
General description:
   Blooming season.—Early March to early April.
   Plant habit.—shrubs (as shown in FIGS. 1, 2 and 5).
Stem description:
   Color of new stem.—tan.
   Stem shape.—cascade (as shown in FIG. 3).
Foliage description:
   Leaf color.—Young leaf: dark purple (FIG. 3); mature leaf: green to dark green (FIG. 3).
   Leaf size.—mature size: about 5.0 cm to about 8.2cm in length and about 1.8 cm to about 2.2 cm in width. (FIG. 3).
   Leaf thickness.—medium.
   Leaf apex.—Sharp (FIG. 3).
   Leaf base.—wedge (FIG. 3).
   Leaf margin.—The margin is jagged.(FIG. 3).
   Leaf shape.—Lanceolate (FIG. 3).
   Leaf pubescence.—absent (FIG. 3).
   Leaf arrangement on stem.—clusters (FIG. 3).
   Leaf venation.—medium.(FIG. 3).
   Leaf texture.—smooth.
   Leaf gloss.—weak.
Petriole description:
   Length.—short.
Flower description:
   Inflorescence.—number of individual flowers per stem: more than 3 (FIG. 4 and 5).
   Individual flower.—Axillary, terminal: axillary or terminal Symmetry: Asymmetry (FIG. 4) Diameter: 2.5-3.5 cm Type: single flower Flower stage: Early March to early April Petals Number: 5 Size: small Shape: oval (FIG. 4) Apex: concave Margin: entire Color: Red Group-52-D Sepals: Shape: oval Color: brown Arrangement: Imbricate structure Stamens Shape: cylindrical Base: connate Pistils Stigma: 3 shallow crack Height: equal or higher than stamens Pubescence on ovary: absent.

What is claimed is:

1. A new and distinct cultivar of Camellia plant named ‘Pink Cascade’ as herein illustrated and described.
